(19) United States
(12) Patent Application Publication
(10) Pub. No.: US 2006/0264855 A1
(43) Pub. Date: Nov. 23, 2006

(54) Method, apparatus and cartridge packing for dosage medicine delivery

(76) Inventors: Boris Goldenberg, New York, NY (US); Ernest S. Geskin, Florham Park, NJ (US); Gordon A. Thomas, Princeton, NJ (US); Robert D. Fechiner, Newark, NJ (US)

Correspondence Address: BORIS GOLDENBERG;#C1008, 370 East 76 Street, New York, NY, 10021, US

(21) Appl. No.: 11/132,594

(22) Filed: May 20, 2005

(51) Int. Cl. A61M 35/00

(52) U.S. Cl. 604/294; 604/290

(57) ABSTRACT

The present invention relates to the contact-less medicine delivery, more particularly to the delivery of medicine via mucosal (oral, nasal, eyeball) for precise, damage-free surface delivery, for more effective penetration of the medicine (vaccine) via mucosal, which is very important for activating immune cells. The method involves accumulating the required amount of the liquid in an elastic tube and expelling this liquid in the direction of the mucosal surface. The fluid is expelled in the course of impact caused by mechanical or electrical forces. This system may be cheap and completely disposable. This fact is very important for mass vaccination. This system can be most effectively used for the delivery of medicine to the eye as one of the body's most sensitive surfaces.

### FIELD OF THE INVENTION

the present invention relates to contact-less medicine delivery, more particularly to the delivery of medicine (e.g., vaccine) via mucosal (oral, nasal, eyeball) for more effective penetration via mucosal, which is very important for activating immune cells. The cost of this system may be cheap and completely disposable. This fact is very important for mass vaccination. This system can be most effectively used for the delivery of medicine to the eye as one of the body's most sensitive surfaces

### BACKGROUND OF THE INVENTION

Medicine delivery via mucosal (oral, nasal, eyeball) constitutes one of the key elements of health care. For example, below we describe medicine delivery to the eye as a very sensitive surface, and the therapy for age-related macular eye degeneration (AMD) alone represents one of the fastest growing areas of the health care industry. Approximately 15 million senior citizens in the U.S. suffer from some form of age-related macular degeneration (AMD). These numbers are estimated to increase as the population ages. Over 2.4 million cataract procedures are performed annually in the US, more than any other therapeutic surgical procedure. This number is projected to increase as a large fraction of the population enters age groups in which the incidence of cataract significantly increases (Macular Degeneration, Canadian Medical Association Journal, Feb. 17, 2004).

Although eye drops have been the standard drug delivery system for over a century, existing technologies of instilling eye drops are not able to meet the demands of the industry. The major concerns in the delivery of eye drops are the accuracy of the direction of the liquid stream, the risk of inadvertent damage to the eye by the dropper bottle contacting the eye, and contamination of the eye and its lids by the patient's helpers' fingers. Currently used eye drop therapies typically provide limited penetration into the front of the eye and virtually no penetration into the back of the eye. Often drops cannot reach the targeted site because of the eye's natural protective surface. In some situations, less than one percent of the medication applied to the surface of the eye actually reaches the targeted site (Drug Delivery for Posterior Segment Eye Disease, Dayle H. Geroski and Henry F. Edelhauser, Department of Ophthalmology, Emory University Eye Center, Atlanta, Georgia)

While the eye drops should be delivered easily and accurately into the eyes in the dosage prescribed, three out of every four people of all ages have difficulty or find it impossible to instill directly from a plastic dropper bottle. Of all forms of medicine packaging, eye drop bottles are the most difficult to use for self-administration purposes. “Instill one drop in eyes three times daily” sounds easy, but for elderly, visually impaired, arthritic or otherwise physically limited persons, efforts to instill the drops frequently prove harrowing and unachievable. As a result, compliance can be compromised and treatment outcome prejudiced. Inefficient methods of drug delivery limit the therapeutic value of ophthalmic medications, and lead to wastage of the medication.

### PREVIOUS ART

A number of delivery systems for eye drops have been suggested. The most common procedure for eye medicine delivery involves squeezing of a flexible bottle targeting an eye. It is assumed that the generated liquid stream will impact the eye surface, thus assuring that the medicine is instilled. Low cost as well as simplicity of operation constitute the obvious advantages of the currently used technology, which enables us to deliver at least a fraction of the liquid expelled from the bottle toward the eye surface. At the same time, control of the amount and the direction of the generated stream are at best insufficient. The duration of the process execution far exceeds the interval between blinks of the eyelids. The liquid is delivered by an incident drop that is inadequately spread over the eye surface, so that only a fraction of the delivered liquid remains on this surface. In the course of administration of the medicine, the dropper bottle can accidentally impact and damage the eye. These and other shortcomings render the process of administering eye medicine using a dropper bottle ineffective. Similar problems exist for nasal and oral medication, especially when the patients are children or animals.

150 patents (30 of them provided recently) have been analyzed. The patents in question can be divided into three groups

1. These patents assist medicine delivery to the eye. One form of such assistance involves the prevention of the closing of the eye lids in the course of the medicine delivery (U.S. Pat. No. 5,382,243, U.S. Pat. No. 5,387,202) Another type of assistance is support of the device and maintenance of the stand off distance during the delivery (U.S. Pat. No. 5,928,662, U.S. Pat. No. 6,398,766). These patents do not provide the metering of the medicine. In addition, there is a requirement to hold the head back during the droplets instilling. Of course, when the head returns to the normal position, the fluid will flow out from the eye surface.

2. Another school of thought suggests decomposing the droplet and forming a spray in order to instill the medicine at the eye surface. The spray is formed by deforming the tube and expelling the liquid via a nozzle attached to the tube (U.S. Pat. No. 6,398,766, U.S. Pat. No. 5,578,021). Another group of patents create a spray by the use of a mini-pump equipped with a nozzle (U.S. Pat. No. 5,588,564, U.S. Pat. No. 5,921,444). An approach similar to perfume bottles is used. These devices enable us to instill the medicine at a normal head position. These devices are reasonably simple but they do not meter the liquid. Some of the known devices (U.S. Pat. No. 5,997,518, U.S. Pat. No. 6,159,188, U.S. Pat. No. 5,881,956) do control the amount of the supplied medicine up to 5 micro liters. These devices are too complicated. In addition, none of the devices of these groups assures the medicine delivery in the course of a time interval less than the interval of blinking.

3. The patents of this group use a spring or storage of mechanical energy in order to expel a desired amount of the fluid at a high rate. These systems are used in ink jet printers. The principal objective of the ink delivery systems, described in a large number of patents, is to minimize the impact zone, which is opposite to the goal of eye medicine delivery devices which should distribute the medicine as evenly as possible over the entire eye surface. There are several (U.S. Pat. No. 5,630,793, U.S. Pat. No. 5,499,751, No. 0 473 892 A2 (European), No. 0 473 892 A3 (European), No. 0 473 892 B1 (European), WO01/34076 A1 (International), U.S. Pat. No. 6,254,579 B1) of this kind of patent concerned with the delivery of eye medicine. All of them, however, are too complicated and fail to control the amount of the delivered medicine in the course of a time interval shorter than the duration of blinking.

The use of a nozzle dramatically reduces the rate of outflow and thus increases the process duration. It seems that it is not possible to reduce process duration down to the blinking time if a nozzle is used. The distribution of the liquid over the surface of the eye is improved by the use of a spray containing fine droplets of the medicine. However, the spray delivery system is not more efficient than the traditional method because the spray consists of both air and liquid, and during delivery, the air blows off some of the liquid. The duration of the drop delivery is more than the interval between blinks.

In our patent the fluid decomposition is attained because of the impact rather than via flow through a nozzle. The precise metering is attained by the control of medicine supply in the elastic tube where the fluid is contained prior to the expulsion, as well as by the diameter of the tube itself. The duration of the fluid removal from the tube is controlled by the energy of the impact. This energy is determined by the compressibility of the spring or by the mass of the striker. The medicine is delivered during a time interval less than the duration of blinking. In addition, it is proposed to supply the medicine in a special capsule similar to those used for oral medicine intake.

A number of new devices for delivery of eye medicine have recently been suggested. None of them, however, is able to assure effective delivery of the medicine. The following conditions must be met by an effective drug delivery system:

1. The complete process duration, beginning from the initiation to the liquid distribution across the eyeball, must be less than the duration between blinks (<0.1 sec).

2. The impact conditions must assure an almost even distribution of the delivered fluid over the mucosal surface.

3. The method must assure speedy droplet decomposition in the course of impact to prevent eye deformation and the generation of stress waves in the eyes.

4. The device can be used without additional sterilization.

5. The control of the device operation (medicine metering with tolerance 0.001 g, device positioning) must be extremely simple and inexpensive.

6. The device has to save medicine.

7. A high fraction of drug penetration must be assured.

8. The device must be affordable.

9. Finally, and most important of all, any possibility of eye damage by impinging droplets or by the device itself must be eliminated.

It is the objective of the present invention to meet the above conditions.

### SUMMARY OF THE INVENTION

The objectives mentioned above can be met by precise metering of a medicine and by rapid decomposition of this medicine into an array of droplets moving toward to the mucosal surface (oral, nasal, eye ball). The duration of the liquid ejection and transit of droplets should be less than 100 msec, and the cross sectional area of the stream should approximately equal, for example, the eye front surface area, while the velocity and size of the droplets as well as the stand off distance should exclude the possibility of eye damage.

According to this invention, the medicine is accumulated in a plastic tube. A moving valve (screw) precisely controls the amount of fluid in the tube. The tube is positioned by a support so that the opening is directed toward the eye. The support is attached to the face and adequately fastened. The fluid is ejected by an impact on the tube (cartridge). The direction of the impact is normal to the direction of the generated microdroplets, and the momentum of the impact is limited. Thus, the impact is sufficient to decompose the fluid into droplets, and to create a stream of droplets directed toward the eye, while the impact of the droplets should not be damaging to the eye.

### BRIEF DESCRIPTION OF DRAWINGS

FIG. 1 shows a schematic plate device for a medicine delivery.

FIGS. 2 and 3 show a schematic piston device for a medicine delivery (cartridge).

FIG. 4 shows a schematic electrical discharge for a medicine delivery.

FIG. 5 shows a schematic of the medicine filling up and welding of the elastic tube .

FIG. 6 shows a schematic piston device for a medicine delivery with a cup holder to keeping eye open.

FIG. 6a, b show a schematic explanation how is a cup holder keeping an eye open

### DETAILED DESCRIPTION OF THE INVENTION

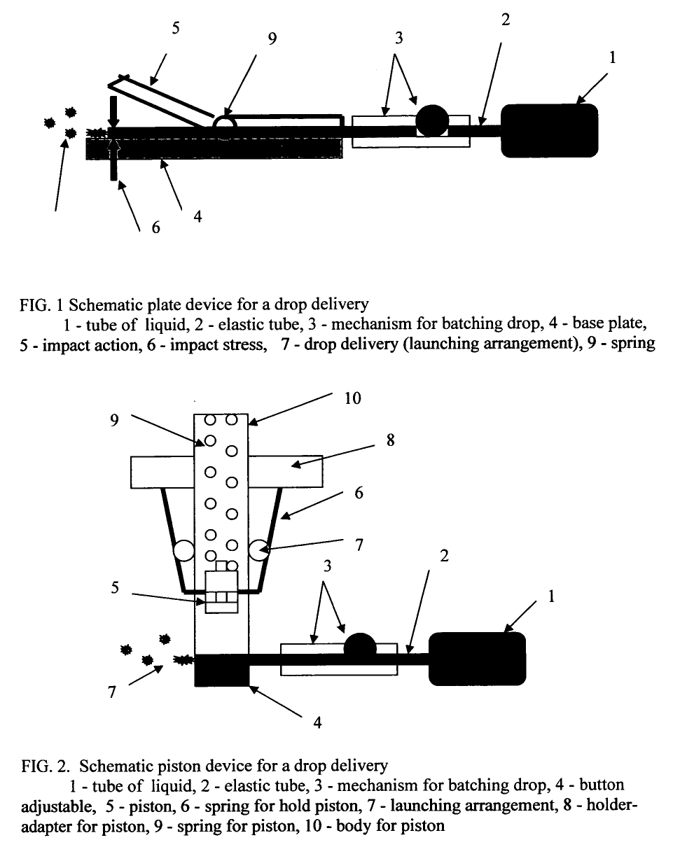

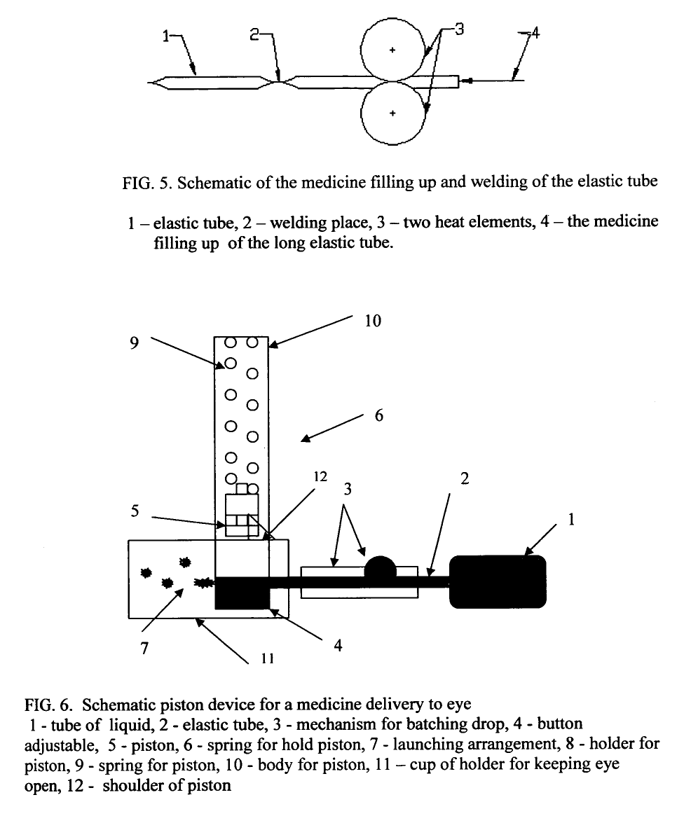

The concept of the method is illustrated by the schematic in FIG. 1. According to this schematic, a dose of a medicine to be delivered to an eye is accumulated in a small elastic tube 2. The small plastic tube could be attached as an extension to a regular plastic bottle 1 or could be produced as a cartridge with a dose of medicine FIG. 5. The impact force is delivered to the tube by a fast moving part 5 driven by a spring 9. As a result, the fluid under the impact stress 6 is expelled 7 from the tube 2 via an open end. The fluid forms an array of the micro droplets 7 driven in the direction of the mucosal surface (nasal, oral or eye). The size and the velocity of droplets are determined by the size of the moving part and by the stiffness and the deformation of the spring, the travel distance of the impacting part, the direction of the impact, and the geometry and elasticity of the tube. The motion of the impacting part is triggered manually or automatically by the release of the spring. The dose of medicine is determined by a moving roll 3 along the tube which pushes the amount of liquid to the open end of the elastic tube 2.

Alternatively, the liquid can be expelled from the elastic tube by the use of electrical forces (Fig. 4). The tube 2 can be squeezed by one or several transducers 5 attached to its wall. The transducers 5 may be triggered by a manual command or automatically.

The direction of the droplets is determined by manual positioning of the tube. The position of the tube can also be determined by a soft support attached to the face of the patient. This support will also be attached to the eyelid by soft links, which will enable us to keep the eyelids open in the course of medicine delivery.

The metering of the liquid is attained by a moving roll or screw 3, which constitutes a partition separating a portion of a medicine to be delivered to the eye from the rest of the fluid, contained in the reservoir. The tube is completely filled by the liquid when the partition is open and completely emptied in the course of the impact. The position of the partition, and thus the amount of the liquid to be delivered, can be precisely controlled manually or automatically.

The mass of the liquid expelled from the tube can also be controlled by the impact conditions. The stiffness of the tube wall, the hydraulic resistance of the tube opening, and fluid pressure in the tube are selected so that the fluid exits the tube only in the course of squeezing of the tube by the impacting part or by the transducer. Thus, the volume of the fluid expelled from the tube is equal to the reduction of the volume of the tube interior.

The procedure of delivery of the eye medicine according to the present invention involves the following steps. The partition is opened and the desired amount of a medicine is inserted into the tube. The tube is connected with a support attached to the face in the vicinity of the eye. The support opens the eyelids and holds them open while the spring is released, the impacting part expels the medicine from the tube, and the micro droplets reach the eye surface. Then the device is removed from the face of the patient and the cycle is repeated.

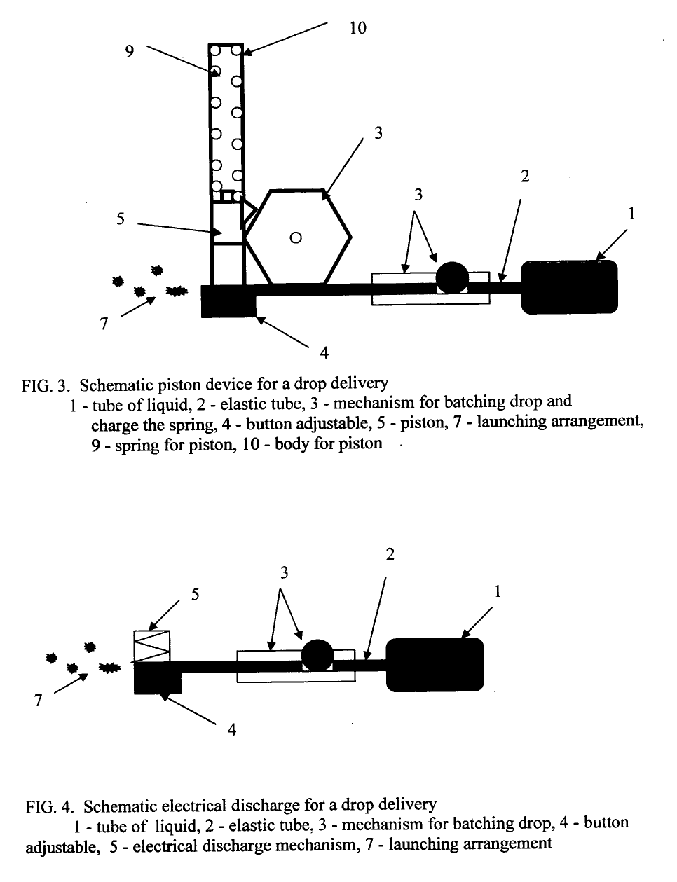

A version of a device implementing the invented method is shown in Fig. 2, Fig. 3 and Fig. 4. Fig. 2 and Fig. 3 show different mechanical discharge devices and Fig. 4 shows the device with electrical impact discharge.

A schematic of the medicine filling up and welding of the elastic tube to the cartridge is shown in Fig. 5. A long elastic tube 1 filling up the medicine through the open end 4 and moving through heated rolls 3 which weld the elastic tube and cut it depend on the required dose of medicine as a cartridge which can be used in a device for dosage medicine delivery.

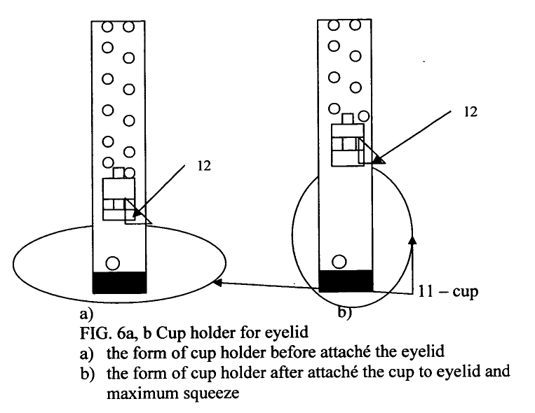

A schematic of the piston device for a medicine delivery to eye is shown in Fig. 6 and FIG. 6a, b. This device has a same principle as show in Fig. 4, but including a cup holder eyelid 11 for keeping eye open. The cup 11 is attached to the button of the body 10 of the piston 5 and the top of the cup has a hole that the top of the cup can move alon the body of the piston. When the operator squeezes the cup 11, the top of the cup moves along the body of the piston and pushes up the piston by the shoulder of the piston 12 and simultaneously holds the eyelids open. When the piston achieves the maximum the hole of the cup increases in size in the direction of the shoulder of the piston and the piston falls down and lunches the medicine directly toward the open eye.

The experiment was conducted with the following parameters: micro tube radius r=0.00127 m, piston spring stiffness coefficient k=775.83 N/m, piston diameter D=0.0172 m, piston weight 0.007 kg and plastic micro tube's stiffness coefficient κₑ₌0.0728 N/m. The resulting diameter of the micro droplet is R=0.000054 m and velocity V₁₌36 m/s. If we take into account that the distance between the eyedrop device and the eyeball ≈25 mm, then the time for drop delivery will be τ=0.00007 s. This time is less than the time it takes to blink, therefore it is good for eye drop delivery. numerical simulation shows that the spring stiffness and also the micro tube radius in the eye drop delivery device can be adjusted very easily. By adjusting these parameters, one could attain different diameters of droplets, as well as different velocities.

What we claim is:

1. The method of the medicine delivery via mucosal surface involving accumulation of a liquid medicine in an elastic tube connected with a reservoir containing the medicine, where the volume of the liquid to be delivered is precisely controlled by a moving partition and is expelled from the elastic tube by an impact sufficient for decomposition of the fluid into the micro droplets and expelled in the direction normal to the direction of the impact.

2. Canceled

3. The method according to claim 1 wherein the reservoir and tube are supported by a part attached to the face of a patient and directing the flow of the generated micro droplets.

4. The method according to claim 1 where the desired amount of the eye medicine is accumulated in a sealed elastic tube and is expelled from this tube as an array of the droplets in the direction of the eye.

5. The method according to claim 1 where the size and the velocity of the micro droplets and the duration of the formation of the micro droplets are determined by the shape and preliminary deformation of the elastic tube, the weight and travel distance of the piston, and the spring compression.

6. The method according to claim 1 where the supply of the fluid into the tube occurs simultaneously with the charging of the spring driving the piston.

7. The method according to claim 1 where tube with the medicine is driven in the direction of the eye and the array of the droplets is formed by the inertia forces generated in the course of the sudden deceleration of the reservoir.

8. The method according to claim 7 where the moving tube opens the eye lids by soft links.

9. The method according to claim 1 where the fluid is expelled from the tube by the electrical force caused by the expansion of a transducer applied to the tube.

10. The method according to claim 1 where the amount of the liquid expelled from the tube is determined by the reduction of the volume of the tube interior in the course of the impact.

11. The device for delivery of an eye medicine containing a reservoir with a liquid medicine connected with an elastic tube having an open end and directed toward the eye where a fast moving piston connected with a spring impacts a section of the tube separated by the movable partition from the reservoir and supported by a rigid platform.

12. The device according to claim 1 where the impacting part is a swinging link.

13. The device according to claim 1 where the sealed tube filled with medicine is installed into the device as a cartridge prior to impact.

14. Device according to claim 1 where the tube is connected with a driving spring and via soft links with the eyelids.

15. Device according to claim 9 where one or several transducers are attached to the wall of the tube.
